(19) United States
(12) Patent Application Publication
(10) Pub. No.: US 2022/0373561 A1
(43) Pub. Date: Nov. 24, 2022

(54) TRANSFORMATIONAL IMMUNE DIAGNOSTICS

(71) Applicant: Arizona Board of Regents on Behalf of Arizona State University, Scottsdale, AZ (US)

(72) Inventor: Stephen Albert JOHNSTON, Tempe, AZ (US)

(21) Appl. No.: 17/816,637

(22) Filed: Aug. 1, 2022

Related U.S. Application Data
(63) Continuation of application No. PCT/US2021/016192, filed on Feb. 2, 2021.
(60) Provisional application No. 62/971,031, filed on Feb. 6, 2020.

(51) Int. Cl. G01N 33/68

(52) U.S. Cl. CPC G01N 33/6854; G01N 33/6845

(57) ABSTRACT

A method of producing a set of high affinity peptides for detecting one or more antibodies associated with a disease or condition of interest. The method including identifying a set of informative peptides correlated to the disease or condition of interest and translating the set of informative peptides to one or more high affinity peptides for an antibody of interest, wherein the presence of the antibody of interest identifies a subject as having the disease or condition of interest. A diagnostic peptide array produced from the one or more high affinity. A method of detecting an antibody associated with a disease of condition, including: contacting a biological sample with a diagnostic peptide array; and detecting the binding of one or more antibodies to a peptide that is associated with a disease or condition of interest, thereby detecting the antibody associated with a disease of condition.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

The present application is a continuation of International Patent Application No. PCT/US2021/016192 filed Feb. 2, 2021, which claims the benefit of United States Provisional Patent Application Ser. No. 62/971,031, filed Feb. 6, 2020, each of which is incorporated herein by reference in its entirety.

### ACKNOWLEDGMENT OF GOVERNMENT SUPPORT

This invention was made with government support under R21 CA220150 awarded by the National Institutes of Health. The government has certain rights in the invention.

### FIELD OF THE DISCLOSURE

This disclosure relates to the field of diagnostics and in particular, to systems and methods for producing immune diagnostics and uses thereof.

### BACKGROUND

One of the great challenges in modern medicine is cost effectively detecting and monitoring diseases and conditions. Early detection of a condition can have a significant impact in the outcome of a disease, and yet, for most conditions, no single test exists that can detect disease before the appearance of major symptoms. Numerous groups have attempted to develop assays that can diagnose specific conditions; however such assays are limited to a specific disease or diagnosis. Moreover, monitoring health over a period of time is cost and time-prohibitive for currently available diagnostic assays.

### SUMMARY

Disclosed herein are methods of producing high affinity peptides for detecting one or more antibodies associated with a disease or condition of interest.

Accordingly, some embodiments provided herein relate to methods of producing a set of high affinity peptides for detecting one or more antibodies associated with a disease or condition of interest. In some embodiments, the methods include identifying a set of informative peptides correlated to the disease or condition of interest and translating the set of informative peptides to one or more high affinity peptides for an antibody of interest. In some embodiments, the presence of the antibody of interest identifies a subject as having the a disease or condition of interest. In some embodiments, identifying a set of informative peptides the method may include contacting a peptide array with a biological sample from an individual with a condition of interest, detecting binding of antibodies in the biological sample with peptides in the peptide array to obtain a first immunosignature profile, and comparing the first immunosignature profile to a control immunosignature profile that is indicative of not having the condition of interest. In some embodiments, differentially bound peptides are identified that either bind less or more antibody in the first immunosignature profile as compared to the control to identify the set of informative peptides correlated to the disease or condition of interest.

Some embodiments provided herein relate to diagnostic peptide arrays. In some embodiments, the arrays are produced from the one or more high affinity peptides for an antibody of interest. In some embodiments, the presence of the antibody of interest identifies a subject as having the disease or condition of interest.

Some embodiments provided herein relate to methods of detecting an antibody associated with a disease of condition. In some embodiments, the methods include contacting a biological sample with a diagnostic peptide array and detecting the binding of one or more antibodies to a peptide in the diagnostic peptide that is associated with a disease or condition of interest. In some embodiments, the antibody associated with a disease of condition is detected.

Some embodiments provided herein relate to methods of producing a set of high affinity peptides for detecting one or more antibodies associated with a disease or condition of interest. In some embodiments, the methods include identifying a set of informative peptides correlated to the disease or condition of interest. In some embodiments, the methods include contacting a peptide array with a biological sample from an individual with a condition of interest, detecting binding of antibodies in the biological sample with the peptide array to obtain a first immunosignature profile, and comparing the first immunosignature profile to a control immunosignature profile that is indicative of not having the condition of interest and identifying differentially bound peptides that either bind less or more antibody in the first immunosignature profile as compared to the control to identify the set of informative peptides correlated to the disease or condition of interest. In some embodiments, the methods include translating the set of informative peptides to one or more high affinity peptides for an antibody of interest, wherein the presence of the antibody of interest identifies a subject as having the disease or condition of interest. In some embodiments, the methods further include contacting a peptide array with a control sample derived from an individual without the known condition, and detecting binding of antibody in the control sample with the peptide array to obtain the control immunosignature profile. In some embodiments, the methods further include constructing a diagnostic peptide array with the one or more high affinity peptides for an antibody of interest. In some embodiments, the methods include repeating any one or more of the steps described herein for a plurality of diseases or conditions of interest to produce one or more high affinity peptides for an antibody of interest for each of the plurality of diseases or conditions of interest.

Some embodiments provided herein relate to diagnostic peptide arrays that are produced using any of the methods described herein. Some embodiments provided herein relate to kits that include any of the diagnostic peptide arrays described herein.

Some embodiments provided herein relate to methods of detecting an antibody associated with a disease of condition. In some embodiments, the methods include contacting a biological sample obtained from a subject with a diagnostic peptide array as described herein, and detecting the binding of one or more antibodies to a peptide that is associated with a disease or condition of interest, thereby detecting the antibody associated with a disease or condition. In some embodiments, the biological sample is serum sample. In some embodiments, the biological sample is a whole blood sample. In some embodiments, the method is used to monitor efficacy of a treatment or reoccurrence.

In some embodiments, the method is used to diagnose a subject with cancer. In some embodiments, the cancer is selected from the group consisting Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloepithelioma, Melanoma, Meningioma, Merkel Cell Carcinoma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple myeloma, Mycosis Fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neurofibroma, Neuroma, Nodular melanoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral cancer, Oropharyngeal Cancer, Osteosarcoma, Ovarian cancer, Ovarian Epithelial Cancer, Ovarian Germ Cell Tumor, Ovarian Low Malignant Potential Tumor, Paget's disease of the breast, Pancoast tumor, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, or Wilms' tumor.

In some embodiments, the method is used to diagnose or prognose a metabolic disease. In some embodiments, the metabolic disease is one or more of abetalipoproteinemia, adrenoleukodystrophy (ALD), crigler-najjar syndrome, cystinuria, hartnup disease, histidinemia, Menkes disease, phenylketonuria (PKU), sitosterolemia, Smith-Lemli-Opiz syndrome, tyrosinemia type I, urea cycle disorders, Wilson's disease, Zellweger syndrome, maple syrup urine disease (MSUD; branched-chain ketoaciduria), glycogen storage disease, glutaric acidemia type 1, alcaptonuria, medium chain acyl dehydrogenase deficiency (glutaric acidemia type 2), acute intermittent porphyria, Lesch-Nhyhan syndrome, congenital adrenal hyperplasia, Kearns-Sayre syndrome, Gaucher's disease, diabetes (type 1), hereditary hemochromatosis, and/or Niemann-Pick disease.

In some embodiments, the method is used to diagnose or prognose a cardiovascular disease. In some embodiments, the cardiovascular disease is one or more of angina, arrhythmia, atherosclerosis, cardiomyopathy, cerebrovascular accident (stroke), cerebrovascular disease, congenital heart disease, Jye Berghofer Syndrome, congestive heart failure, myocarditis, valve disease, coronary artery disease, dilated cardiomyopathy, diastolic dysfunction, endocarditis, high blood pressure (hypertension), hypertrophic cardiomyopathy, mitral valve prolapse, myocardial infarction, and/or venous thromboembolism.

In some embodiments, the method is used to diagnose or prognose a dermatological disorders. In some embodiments, the dermatological disorder is one or more of acne, actinic keratosis, angioma, Athlete's foot, aquagenic pruritus, argyria, atopic dermatitis, baldness, basal cell carcinoma, bed sore, Behcet's disease, blepharitis, boil, Bowen's disease, bullous pemphigoid, canker sore, carbuncles, cellulitis, chloracne, chronic dermatitis of the hands and feet, cold sores, contact dermatitis (includes poison ivy, oak, sumac), creeping eruption, dandruff, dermatitis, dermatitis herpetiformis, dermatofibroma, diaper rash, dyshidrosis, eczema, epidermolysis bullosa, erysipelas, erythroderma, friction blister, genital wart, gestational pemphigoid, Grover's disease, hemangioma, Hidradenitis suppurativa, hives, hyperhidrosis, ichthyosis, impetigo, jock itch, Kaposi's sarcoma, keloid, keratoacanthoma, keratosis pilaris, Lewandowsky-Lutz dysplasia, lice infection, Lichen planus, Lichen simplex chronicus, lipoma, lymphadenitis, malignant melanoma, melasma, miliaria, molluscum contagiosum, nummular dermatitis, Paget's disease of the nipple, pediculosis, pemphigus, perioral dermatitis, photoallergy, photosensitivity, Pityriasis rosea, Pityriasis rubra pilaris, porphyria, psoriasis, Raynaud's disease, ringworm, rosacea, scabies, scleroderma, scrofula, sebaceous cyst, seborrheic keratosis, seborrhoeic dermatitis, shingles, skin cancer, skin tags, spider veins, squamous cell carcinoma, stasis dermatitis, sunburn, tick bite, tinea barbae, tinea capitis, tinea corporis, tinea cruris, tinea pedis, tinea unguium, tinea versicolor, tinea, tungiasis, urticaria (Hives), Vagabond's disease, vitiligo, warts, and/or wheal (“weal” and “welt”).

In some embodiments, the method is used to diagnose or prognose a hematological disorders. In some embodiments, the hematological disorders is one or more of Anaphylactoid Purpura (Henock-Schönlein Disease), allergic purpura (Henock-Schönlein Disease), low red blood cells (anemia), hemolytic anemia, hypoproliferative anemia, macrocytic anemia, microcytic anemia, normocytic anemia, pernicious anemia (Vitamin B12 deficiency), basophilia, blood vessel abnormalities, dysfibrinogenemia, eosinophilia, erythrocytosis/polycythemia, essential thrombocythemia, excess platelets (thrombocytosis), excess red blood cells (erythrocytosis/polycythemia), excess white blood cells (leukocytosis), Factor V Leiden Mutation, fibrin clot formation abnormalities, folic acid deficiency, hemophilia, hereditary von Willebrand's Disease, inherited hypercoagulation disorders, inherited platelet abnormalities, iron deficiency, low platelets (thrombocytopenia), low white blood cells (neutropenia), lymphocytosis, myelofibrosis with myeloid metaplasia, monocytosis, myeloproliferative disorders, neutrophilia, platelet abnormalities, polycythemia vera, premalignant blood disorders, scurvy, Systemic Lupus Erythematosus (SLE), thrombocytopenia, and/or sickle cell disease.

In some embodiments, the method is used to diagnose or prognose a neurodegenerative disease. In some embodiments, the neurodegenerative disease is one or more of alcoholism, Alexander's disease, Alper's disease, Alzheimer's disease, Amyotrophic lateral sclerosis, ataxia telangiectasia, Batten disease (also known as Spielmeyer-Vogt-Sjogren-Batten disease), bovine spongiform encephalopathy (BSE), Canavan disease, Cockayne syndrome, Corticobasal degeneration, Creutzfeldt-Jakob disease, Huntington's disease, HIV-associated dementia, Kennedy's disease, Krabbe's disease, Lewy body dementia, Machado-Joseph disease (Spinocerebellar ataxia type 3), multiple sclerosis, Multiple System Atrophy, narcolepsy, neuroborreliosis, Parkinson's disease, Pelizaeus-Merzbacher Disease, Pick's disease, primary lateral sclerosis, prion diseases, Refsum's disease, Sandhoff's disease, Schilder's disease, subacute combined degeneration of spinal cord secondary to pernicious anaemia, schizophrenia, spinocerebellar ataxia (multiple types with varying characteristics), spinal muscular atrophy, Steele-Richardson-Olszewski disease, and/or Tabes dorsalis.

In some embodiments, the method is used to diagnose or prognose an inflammatory disease. In some embodiments, the inflammatory disease is one or more of asthma, autoimmune diseases, chronic inflammation, chronic prostatitis, glomerulonephritis, hypersensitivities, inflammatory bowel diseases, pelvic inflammatory disease, reperfusion injury, rheumatoid arthritis, transplant rejection, and/or vasculitis.

The foregoing and other features of the disclosure will become more apparent from the following detailed description, which proceeds with reference to the accompanying figures.

### BRIEF DESCRIPTION OF THE DRAWINGS

FIG. 1 is a diagram of an exemplary transformational immune diagnostics method.

FIG. 2 depicts array peptide analyses, depicting four mimotopes selected from the immunosignature (IMS) array analysis (left). Antibodies purified by these mimotopes showed specific reactivity to the target antigen (right).

FIG. 3 depicts array peptide analyses, depicting informative peptides selected from the immunosignature (IMS) analysis (left). The “ADSxx” epitope was identified from these informative peptides (right).

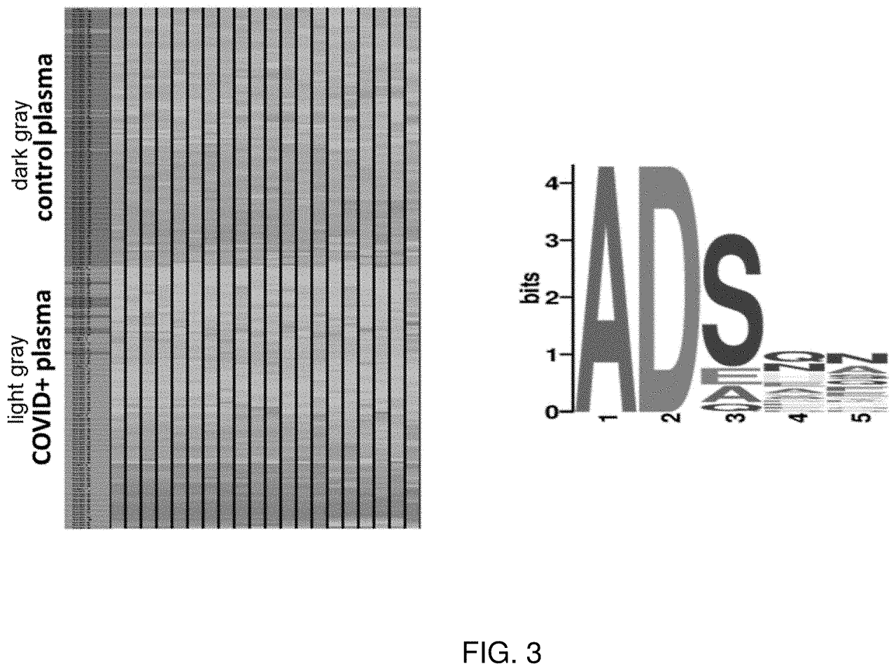

### DETAILED DESCRIPTION

In the following detailed description, reference is made to the accompanying drawings which form a part hereof, and in which are shown by way of illustration embodiments that may be practiced. It is to be understood that other embodiments may be utilized and structural or logical changes may be made without departing from the scope. Therefore, the following detailed description is not to be taken in a limiting sense, and the scope of embodiments is defined by the appended claims and their equivalents.

Various operations may be described as multiple discrete operations in turn, in a manner that may be helpful in understanding embodiments; however, the order of description should not be construed to imply that these operations are order dependent.

For the purposes of the description, a phrase in the form “A/B” or in the form “A and/or B” means (A), (B), or (A and B). For the purposes of the description, a phrase in the form “at least one of A, B, and C” means (A), (B), (C), (A and B), (A and C), (B and C), or (A, B and C). For the purposes of the description, a phrase in the form “(A)B” means (B) or (AB) that is, A is an optional element.

The description may use the terms “embodiment” or “embodiments,” which may each refer to one or more of the same or different embodiments. Furthermore, the terms “comprising,” “including,” “having,” and the like, as used with respect to embodiments, are synonymous, and are generally intended as “open” terms (e.g., the term “including” should be interpreted as “including but not limited to,” the term “having” should be interpreted as “having at least,” the term “includes” should be interpreted as “includes but is not limited to,” etc.).

With respect to the use of any plural and/or singular terms herein, those having skill in the art can translate from the plural to the singular and/or from the singular to the plural as is appropriate to the context and/or application. The various singular/plural permutations may be expressly set forth herein for sake of clarity.

Unless otherwise noted, technical terms are used according to conventional usage. Definitions of common terms in molecular biology can be found in Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); and Robert A. Meyers (ed.), Molecular Biology and Biotechnology: a Comprehensive Desk Reference, published by VCH Publishers, Inc., 1995 (ISBN 9780471185710); and other similar references. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

To facilitate review of the various embodiments of this disclosure, the following explanations of specific terms are provided, along with particular examples:

Antigen: A compound, composition, or substance that can stimulate the production of antibodies or a T cell response in an animal, including compositions that are injected or absorbed into an animal. An antigen reacts with the products of specific humoral or cellular immunity, including those induced by heterologous immunogens, such as the peptides disclosed herein. The term “antigen” includes all related antigenic epitopes. “Epitope” or “antigenic determinant” refers to a site on an antigen to which B and/or T cells respond. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 amino acids in a unique spatial conformation. Methods of determining spatial conformation of epitopes include, for example, x-ray crystallography and 2-dimensional nuclear magnetic resonance.

Antibody: Immunoglobulin molecules and immunologically active portions of immunoglobulin molecules, i.e., molecules that contain an antigen binding site that specifically binds (immunoreacts with) an antigen.

A naturally occurring antibody (e.g., IgG, IgM, IgD) includes four polypeptide chains, two heavy (H) chains and two light (L) chains interconnected by disulfide bonds. However, it has been shown that the antigen-binding function of an antibody can be performed by fragments of a naturally occurring antibody. Thus, these antigen-binding fragments are also intended to be designated by the term “antibody.” Specific, non-limiting examples of binding fragments encompassed within the term antibody include (i) a Fab fragment consisting of the VL, VH, CL and CH1 domains; (ii) an Fd fragment consisting of the VH and CH1 domains; (iii) an Fv fragment consisting of the VL and VH domains of a single arm of an antibody, (iv) a dAb fragment (Ward et al., Nature 341:544-546, 1989) which consists of a VH domain; (v) an isolated complementarity determining region (CDR); and (vi) a F(ab′)2 fragment, a bivalent fragment comprising two Fab fragments linked by a disulfide bridge at the hinge region.

Immunoglobulins and certain variants thereof are known and many have been prepared in recombinant cell culture (e.g., see U.S. Pat. Nos. 4,745,055; 4,444,487; WO 88/03565; EP 256,654; EP 120,694; EP 125,023; Faoulkner et al., Nature 298:286, 1982; Morrison, J. Immunol. 123:793, 1979; Morrison et al., Ann Rev Immunol 2:239, 1984). Humanized antibodies and fully human antibodies are also known in the art.

Animal: Living multi-cellular vertebrate organisms, a category that includes, for example, mammals and birds. The term mammal includes both human and non-human mammals. Similarly, the term “subject” includes both human and veterinary subjects.

Array: An arrangement of molecules, such as biological macromolecules (such as peptides), in addressable locations on or in a substrate. A “microarray” is an array that is miniaturized so as to require or be aided by microscopic examination for evaluation or analysis. The array of molecules (“features”) makes it possible to carry out a very large number of analyses on a sample at one time. Within an array, each arrayed sample is addressable, in that its location can be reliably and consistently determined within at least two dimensions of the array. The feature application location on an array can assume different shapes. For example, the array can be regular (such as arranged in uniform rows and columns) or irregular. Thus, in ordered arrays the location of each sample is assigned to the sample at the time when it is applied to the array, and a key may be provided in order to correlate each location with the appropriate target or feature position. Often, ordered arrays are arranged in a symmetrical grid pattern, but samples can be arranged in other patterns (such as in radially distributed lines, spiral lines, or ordered clusters). Addressable arrays usually are computer readable, in that a computer can be programmed to correlate a particular address on the array with information about the sample at that position (such as hybridization or binding data, including for instance signal intensity). In some examples of computer readable formats, the individual features in the array are arranged regularly, for instance in a Cartesian grid pattern, which can be correlated to address information by a computer.

Binding or stable binding: An association between two substances or molecules, such as the association of an antibody with a peptide. Binding can be detected by any procedure known to one skilled in the art, such as by physical or functional properties of the formed complexes, such as a target/antibody complex.

Diagnostic: Identifying the presence or nature of a pathologic condition. Diagnostic methods differ in their sensitivity and specificity. The “sensitivity” of a diagnostic assay is the percentage of diseased individuals who test positive (percent of true positives). The “specificity” of a diagnostic assay is 1 minus the false positive rate, where the false positive rate is defined as the proportion of those without the disease who test positive. While a particular diagnostic method may not provide a definitive diagnosis of a condition, it suffices if the method provides a positive indication that aids in diagnosis. “Prognostic” means predicting the probability of development (for example, severity) of a pathologic condition.

Immune response: A response of a cell of the immune system, such as a B cell, T cell, or monocyte, to a stimulus. In one embodiment, the response is specific for a particular antigen (an “antigen-specific response”).

Immunogenic peptide: A peptide which comprises an allele-specific motif or other sequence such that the peptide will bind an MHC molecule and induce a cytotoxic T lymphocyte (“CTL”) response, or a B cell response (e.g. antibody production) against the antigen from which the immunogenic peptide is derived.

Immunosignature: A statistically significant pattern of peptides, each with specific binding values that can robustly classify one state of disease from others.

Immunosignature profile: A profile that is specific for a particular condition or disease based upon the immunosignature.

Informative peptide: A peptide that is identified as binding to an antibody associated with, or indicative of a particular disease or condition.

Label: A detectable compound or composition that is conjugated directly or indirectly to another molecule to facilitate detection of that molecule. Specific, non-limiting examples of labels include fluorescent tags, enzymatic linkages, and radioactive isotopes.

Peptide Modifications: Immunogenic peptides include synthetic embodiments of peptides described herein. In addition, analogs (non-peptide organic molecules), derivatives (chemically functionalized peptide molecules obtained starting with the disclosed peptide sequences) and variants (homologs) of these proteins can be utilized in the methods described herein. Each polypeptide of this disclosure is comprised of a sequence of amino acids, which may be either L- and/or D- amino acids, naturally occurring and otherwise.

Peptides can be modified by a variety of chemical techniques to produce derivatives having essentially the same activity as the unmodified peptides, and optionally having other desirable properties. For example, carboxylic acid groups of the protein, whether carboxyl-terminal or side chain, can be provided in the form of a salt of a pharmaceutically-acceptable cation or esterified to form a C1-C16 ester, or converted to an amide of formula NR1R2 wherein R1 and R2 are each independently H or C1-C16 alkyl, or combined to form a heterocyclic ring, such as a 5- or 6- membered ring Amino groups of the peptide, whether amino-terminal or side chain, can be in the form of a pharmaceutically-acceptable acid addition salt, such as the HCl, HBr, acetic, benzoic, toluene sulfonic, maleic, tartaric and other organic salts, or can be modified to C1-C16 alkyl or dialkyl amino or further converted to an amide.

Hydroxyl groups of the peptide side chains may be converted to C1-C16 alkoxy or to a C1-C16 ester using well-recognized techniques. Phenyl and phenolic rings of the peptide side chains may be substituted with one or more halogen atoms, such as fluorine, chlorine, bromine or iodine, or with C1-C16 alkyl, C1-C16 alkoxy, carboxylic acids and esters thereof, or amides of such carboxylic acids. Methylene groups of the peptide side chains can be extended to homologous C2-C4 alkylenes. Thiols can be protected with any one of a number of well-recognized protecting groups, such as acetamide groups. Those skilled in the art will also recognize methods for introducing cyclic structures into the peptides of this disclosure to select and provide conformational constraints to the structure that result in enhanced stability.

Peptidomimetic and organomimetic embodiments are envisioned, whereby the three-dimensional arrangement of the chemical constituents of such peptido- and organomimetics mimic the three-dimensional arrangement of the peptide backbone and component amino acid side chains, resulting in such peptido- and organomimetics of an immunogenic Brachyury polypeptide having measurable or enhanced ability to generate an immune response. For computer modeling applications, a pharmacophore is an idealized three-dimensional definition of the structural requirements for biological activity. Peptido- and organomimetics can be designed to fit each pharmacophore with current computer modeling software (using computer assisted drug design or CADD). See Walters, “Computer-Assisted Modeling of Drugs,” in Klegerman & Groves, eds., 1993, Pharmaceutical Biotechnology, Interpharm Press: Buffalo Grove, Ill, pp. 165-174 and Principles of Pharmacology, Munson (ed.) 1995, Ch. 102, for descriptions of techniques used in CADD. Also included are mimetics prepared using such techniques.

Peptide: Any chain of amino acids, regardless of length or post-translational modification (e.g., glycosylation or phosphorylation). A polypeptide can be between 3 and 30 amino acids in length. In one embodiment, a polypeptide is from about 5 to about 25 amino acids in length. In yet another embodiment, a polypeptide is from about 8 to about 12 amino acids in length. In yet another embodiment, a peptide is about 5 amino acids in length. With regard to polypeptides, the word “about” indicates integer amounts.

Sequence identity: The similarity between amino acid sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a polypeptide will possess a relatively high degree of sequence identity when aligned using standard methods.

Within the context of an immunogenic peptide, a “conserved residue” is one which appears in a significantly higher frequency than would be expected by random distribution at a particular position in a peptide. In one embodiment, a conserved residue is one where the MHC structure may provide a contact point with the immunogenic peptide.

Methods of alignment of sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. USA 85:2444, 1988. Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, Md.) and on the internet, for use in connection with the sequence analysis programs blastp, blastn, blastx, tblastn and tblastx. A description of how to determine sequence identity using this program is available on the NCBI website on the internet.

Homologs and variants of a polypeptide are typically characterized by possession of at least 75%, for example at least 80%, sequence identity counted over the full length alignment with the amino acid sequence using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 80%, at least 85%, at least 90%, at least 95%, at least 98%, or at least 99% sequence identity. Methods for determining sequence identity over such short windows are available at the NCBI website on the internet. One of skill in the art will appreciate that these sequence identity ranges are provided for guidance only; it is entirely possible that strongly significant homologs could be obtained that fall outside of the ranges provided.

Suitable methods and materials for the practice or testing of this disclosure are described below. Such methods and materials are illustrative only and are not intended to be limiting. Other methods and materials similar or equivalent to those described herein can be used. For example, conventional methods well known in the art to which this disclosure pertains are described in various general and more specific references, including, for example, Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999; Harlow and Lane, Antibodies: A Laboratory Manual, Cold Spring Harbor Laboratory Press, 1990; and Harlow and Lane, Using Antibodies: A Laboratory Manual, Cold Spring Harbor Laboratory Press, 1999. In addition, the materials, methods, and examples are illustrative only and not intended to be limiting.

### Description of Several Embodiments

### Introduction

Immunosignatures based on using peptides chosen from random sequence space have been shown to be broadly applicable to diagnose a wide variety of disease early. However, the manufacture of such peptide arrays and their use as a diagnostic is problematic because the of the low affinity of the antibodies to mimotopes. Embodiments provided herein relate to methods, systems, and devices that allow the translation of the immunosignatures into a more useful high affinity antibody assay.

Immunosignature technology has unique advantages for diagnostics. It involves making arrays with peptides chosen from random sequence space to optimally present chemical diversity. This allows splaying out the diversity of the antibodies in the blood or other biological samples with amazing resolution. Only a drop of blood, saliva etc. is required. Because antibodies are amplified in response to disease, disease can be detected early. Immunosignatures were designed to take advantage of this antibody response (see WO 2014036312 which is hereby incorporated by reference in its entirety for all aspects relating to immunosignaturing and peptide arrays and methods of use thereof). Because the peptides used in immunosignaturing are generated from random sequence space, there is little bias in the antibodies detected. For example, the antibodies could originate by being activated by any type of antigen. Immunosignaturing is especially sensitive relative to, for example, an ELISA. This is because the non-informative peptides buffer out the non-specific antibody competition. In addition, unlike in an ELISA which sums signal from both specific and non-specific binding, immunosignaturing only detects the specific antibody bound by the peptides of interest.

However, there are some limitations in immunosignaturing. While using random sequence space peptides affords an unprecedented sensitivity to discover new diagnostic peptides, it also requires binding the antibodies based on avidity. This is because the affinity for the peptides is relatively weak, so they have to be spaced close enough to incur avidity. Since the immune signature depends on detecting small differences in binding of millions of antibodies, the system is very sensitive to changes in conditions. This includes the nature of the array, requiring very demanding production capabilities to have reproducible diagnostics. These and other issues may inhibit the use of the immunosignaturing process in fast and/or fieldable assays.

Embodiments of the present disclosure relate to systems, devices, and methods that solve the aforementioned and other problems. In some embodiments, the systems, devices, and methods relate to processes and arrays that take full advantage of the unique features of immunosignaturing, and combines these advantages with a new type of array that will solve the limitations of immunosignaturing. This technological solution is termed herein Transformational Immune Diagnostic System (TIDS).

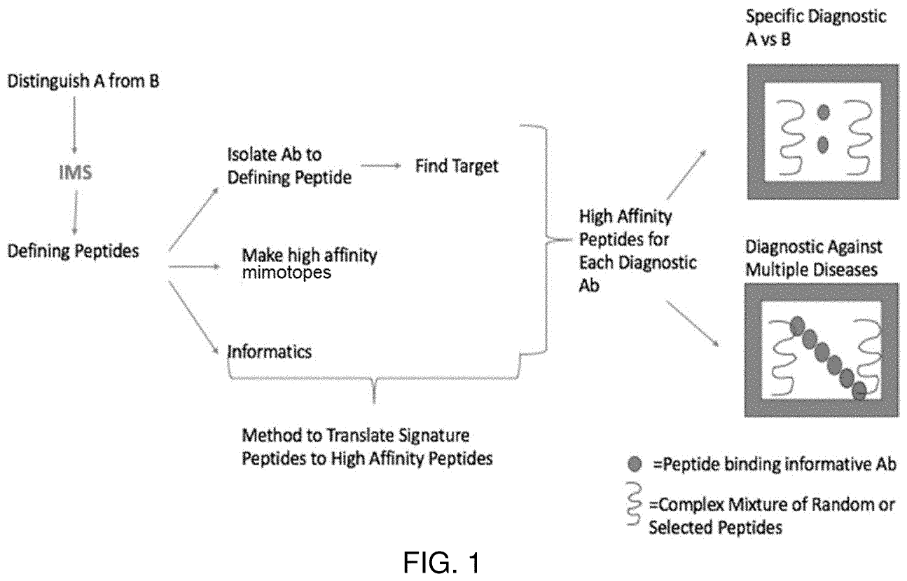

In exemplary embodiments, such as illustrated in FIG. 1, TIDS comprises determining and/or detecting peptides that bind one or more antibodies of interest, for example, one or more antibodies that are correlated to a particular disease and/or condition (which can also be known as a signature or informative antibody). There are multiple approaches to constructing a set of peptides that bind an antibody of interest. An important point is that because these arrays are just being used in the discovery of informative antibodies, the arrays can be optimized for discovery (larger size, number of peptides etc.) without the constraints of trying to use them in diagnosis. The random sequence arrays used in immunosignatures (IMS) have been shown to work very well in this process. However, other types of arrays could also be used in the discovery step.

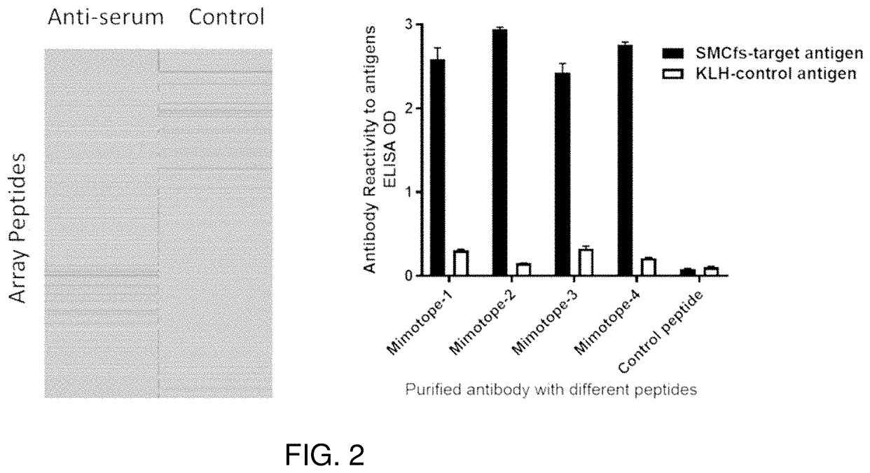

In one embodiment, informative antibodies are affinity purified using the random peptides they bind. It may also be possible in some cases to reverse engineer the antibody using the peptide sequence, which may be graphed, for example, by nucleic acid manipulation into an antibody scaffold. The antibody can then be used to probe the relevant cellular material to find the original antigen target, for example, by combining a pull down or Western blot with mass spectrometry. Once the target has been found, it can be used to determine the actual peptide sequence that induced the informative antibody. It is contemplated that such a peptide would be of high affinity for the informative antibody. As an example, in FIG. 2, mimotopes are screened to a target antigen from IMS array and purified specific antibodies.

In one embodiment, a peptide, for example, a mimotope, is derived that has higher affinity for the informative antibody than an informative random peptide from the array. This can be done by creating a library of variants on the IMS random peptide and selecting or screening for those with the highest affinity. This sequence does not have to correspond to the natural sequence of the inducing antigen, but has high affinity for the informative antibody. An advantage of this method is that the target antigen and/or epitope does not need to be identified. As an example, informative peptides were selected that have significantly reactive to antibodies in COVID patients from the IMS analysis. The epitope “ADSxx” from these informative peptides does not match to any known immunogenic antigens of the SARS-CoV2.

Another important advantage is that these high affinity antibodies can be spaced (e.g., >3 nm apart) to avoid background binding due to avidity (compare Compound Arrays for Sample Profiling, U.S. Pat. No. 10,422,793). Such spacing provides for a more robust assay.

In one embodiment, a peptide, for example, that has high affinity for the informative antibody is determined by informatic induction. For example, by aligning the sequences in the informative peptides potential epitope targets can be identified. These potential epitope targets can be matched against proteome sequences to find the original antigen and the natural high affinity peptide identified. Alternatively, the sequences of the potential epitope targets can be used as described above to define a high affinity peptide. However, in all of the methods described the goal is to assemble a collection of peptides that would bind all the antibodies that formed the original IMS defining diagnostic. These peptides would be high affinity to the informative antibody.

The second phase of the TIDS relies on a new type of array design. It allows for simple, robust production of arrays that can be used in an ELISA-like format but has the sensitivity of IMS. In some embodiments, the high affinity peptides for the signature antibody, or signature antibodies of interest if more than one disease, are spotted on a surface in an addressable fashion. In some embodiments, these peptides are pre-made and do not require in-situ synthesis. In some embodiments, the remainder of the array is then covered by a complex collection of random sequence peptides or peptides chosen not to bind the signature antibodies. This complex mixture provides competition for non-signature antibodies to bind and increases the sensitivity. In some embodiments, there are multiple sets of high affinity, diagnostic peptides for all diseases of interest. This arrangement allows for doing disease monitoring for early detection. In some embodiments, the disclosed arrays are produced on a massive scale.

In some embodiments, a biological sample, such as blood, saliva etc. is applied to a disclosed array and allowed to incubate in any ELISA format. Following incubation, the array is washed and the amount of antibody on the high affinity, diagnostic peptides measured. In some embodiments, the amount of antibody on the high affinity diagnostic peptides is measured indirectly, such as by labeling with a secondary antibody or by direct measures. It is only necessary to read the informative spots. Unlike an ELISA, the informative spots are not summed with the background. Because high affinity binding is being detected the assay is fast and lends itself to be fieldable or in the home.

As shown in FIG. 1, the assay system is depicted as an array with a buffer from a complex mixture of random peptides. However, other types of formats are possible, as well as other types of buffering peptides.

### Methods of Producing Peptide Arrays for the Detection of Disease or Condition

Immunosignaturing is a peptide array-based technology that quantitates the dynamics of circulating antibodies in a subject. The dynamics of circulating antibodies includes both the presence and the absence of an antibody or a plurality of antibodies from the system of a subject. The method is based on the sensitivity of the antibody profile in an individual to the development of aberrant cells. Even a small number of initiating cancer or other diseased cells can initiate a B-cell response that can be amplified 10¹¹ fold in a week.

Disclosed herein is a method of producing a set of high affinity peptides for detecting one or more antibodies that are associated with one or more diseases or conditions of interest, for example as implemented in a peptide array. In some embodiments, method of producing a set of high affinity peptides for detecting one or more antibodies that are associated with one or more diseases or conditions of interest includes identifying a signature peptide profile for a disease or condition of interest, such as a set of informative peptides correlated to the disease or condition of interest, and translating the signature peptide profile to one or more high affinity peptides for an antibody of interest, wherein the presence of the antibody of interest identifies a subject as having the a disease or condition of interest. In embodiments, identifying a signature peptide profile includes: contacting a peptide array with a first biological sample, such as sera, from an individual with a condition of interest; detecting binding of antibodies in the first biological sample with peptides in the peptide array to obtain a first immunosignature profile; comparing the first immunosignature profile to a control immunosignature profile that is indicative of not having the condition of interest; and identifying differentially bound peptides from the peptide array as compared to the control to construct the signature peptide profile. By splaying the antibody repertoire out on an array of peptides (immunosignaturing) and comparing disease or condition to a normal control a set of informative peptides (that is peptides that are identified as binding to antibodies associated with, or indicative of the disease or condition) can be identified. The differentially binding peptides can be identified by any suitable method, including but not limited to sequence analysis, comparison to the proteome, prior knowledge of peptides at particular positions on the array. For example, the informative peptides can be identified with informatics methods. Arrays consisting of peptides corresponding to human protein sequences can be directly aligned with the epitope in the corresponding protein. Random peptide arrays and non-random or pseudo-random peptide arrays can be used to identify and align peptide sequences to human proteins. In comparing disease to non-disease samples, a large number of peptides that bind more antibody in the non-disease than the disease samples can be identified. In embodiments, identifying differentially bound peptides incudes identifying peptides on the peptide array that either bind less or more antibody in the first immunosignature profile as compared to the control. The control can be any suitable control. In one embodiment, the control comprises non-disease biological sample, such as sera, contacted with an identical array under the same experimental conditions. The control can be values taken form such a control, such that the control and test need not be conducted at the same time. Comparison of the disease immune profile to a normal control and identifying differentially bound peptides can be carried out via any suitable technique. In some embodiments, the method incudes contacting a peptide array with a control sample derived from an individual without the known condition and detecting binding of antibody in the control sample with the peptide array to obtain a second immunosignature profile and comparing this second immunosignature profile to the first immunosignature profile to identify differentially bound peptides from the peptide array as compared to the control. In some examples, control sample is obtained from multiple individuals. For example sera obtained from healthy volunteers display a rather broad distribution of baseline binding reactivity, thus sample sera from a large number of non-diseased individuals may accommodate the population variability. In addition, signatures from sera of persons with a given disease are extremely consistent, unlike that of the non-disease sera. This observation implies that the immune system is constantly probing and reacting to local environments causing broad differences in signatures. However, once directed toward an antigen, antibodies tend to form a narrow and well-defined signature with little individual variability. In some embodiments, the control is a set of control values, for example an average or even weighted average, of the immunosignature profile of several healthy individuals.

The binding of an antibody to a peptide array creates a pattern of binding that can be associated with a condition. The affinity of binding of an antibody to a peptide in the array can be mathematically associated with a condition. The binding pattern of an antibody to a plurality of different peptides of a peptide array can be mathematically associated with a condition. The avidity of binding of an antibody to a plurality of different peptides of a peptide array can be mathematically associated with a condition. This binding and avidity can comprise the interaction of an antibody in a biological sample with multiple, non-identical peptides in a peptide array. An avidity of binding of an antibody with multiple, non-identical peptides in a peptide array can determine an association constant of the antibody to the peptide array. In some embodiments, the concentration of an antibody in a sample contributes to an avidity of binding to a peptide array, for example, by trapping a critical number or antibodies in the array and allowing for rapid rebinding of an antibody to an array.

The avidity of binding of an antibody to a peptide array can be determined by a combination of multiple bond interaction. A cross-reactivity of an antibody to multiple peptides in a peptide array can contribute to an avidity of binding. In some embodiments, an antibody can recognize an epitope of about 3 amino acids, about 4 amino acids, about 5 amino acids, about 6 amino acids, about 7 amino acids, about 8 amino acids, about 9 amino acids, about 10 amino acids, about 11 amino acids, about 12 amino acids, about 13 amino acids, about 14 amino acids, about 15 amino acids, about 16 amino acids, or about 17 amino acids. In some embodiments, a sequence of about 5 amino acids dominates a binding energy of an antibody to a peptide.

Off-target binding, and/or avidity, of an antibody to a peptide within a peptide array, for example, effectively compresses binding affinities that span femtomolar (fM) to micromolar (μM) dissociation constants into a range that can be quantitatively measured using only 3 logs of dynamic range. An antibody can bind to a plurality of peptides in the array with association constants of 10³ M⁻¹ or higher. An antibody can bind to a plurality of peptides in the array with association constants ranging from 10³ to 10⁶ M⁻¹, 2×10³ M⁻¹ to 10⁶ M⁻¹, and/or association constants ranging from 10⁴ M⁻¹ to 10⁶ M⁻¹. An antibody can bind to a plurality of peptides in the array with a dissociation constant of about 1 fM, about 2 fM, about 3 fM, about 4 fM, about 5 fM, about 6 fM, about 7 fM, about 8 fM, about 9 fM, about 10 fM, about 20 fM, about 30 fM, about 40 fM, about 50 fM, about 60 fM, about 70 fM, about 80 fM, about 90 fM, about 100 fM, about 200 fM, about 300 fM, about 400 fM, about 500 fM, about 600 fM, about 700 fM, about 800 fM, about 900 fM, about 1 picomolar (pM), about 2 pM, about 3 pM, about 4 pM, about 5 pM, about 6 pM, about 7 pM, about 8 pM, about 9 pM, about 10 pM, about 20 pM, about 30 pM, about 40 pM, about 50 pM, about 60 pM, about 7 pM, about 80 pM, about 90 pM, about 100 pM, about 200 pM, about 300 pM, about 400 pM, about 500 pM, about 600 pM, about 700 pM, about 800 pM, about 900 pM, about 1 nanomolar (nM), about 2 nM, about 3 nM, about 4 nM, about 5 nM, about 6 nM, about 7 nM, about 8 nM, about 9 nM, about 10 nM, about 20 nM, about 30 nM, about 40 nM, about 50 nm, about 60 nM, about 70 nM, about 80 nM, about 90 nM, about 100 nM, about 200 nM, about 300 nM, about 400 nM, about 500 nM, about 600 nM, about 700 nM, about 800 nM, about 900 nM, about 1 μM, about 2 μM, about 3 μM, about 4 μM, about 5 μM, about 6 μM, about 7 μM, about 8 μM, about 9 μM, about 10 μM, about 20 μM, about 30 μM, about 40 μM, about 50 μM, about 60 μM, about 70 μM, about 80 μM, about 90 μM, or about 100 μM.

An antibody can bind to a plurality of peptides in the array with a dissociation constant of at least 1 fM, at least 2 fM, at least 3 fM, at least 4 fM, at least 5 fM, at least 6 fM, at least 7 fM, at least 8 fM, at least 9 fM, at least 10 fM, at least 20 fM, at least 30 fM, at least 40 fM, at least 50 fM, at least 60 fM, at least 70 fM, at least 80 fM, at least 90 fM, at least 100 fM, at least 200 fM, at least 300 fM, at least 400 fM, at least 500 fM, at least 600 fM, at least 700 fM, at least 800 fM, at least 900 fM, at least 1 picomolar (pM), at least 2 pM, at least 3 pM, at least 4 pM, at least 5 pM, at least 6 pM, at least 7 pM, at least 8 pM, at least 9 pM, at least 10 pM, at least 20 pM, at least 30 pM, at least 40 pM, at least 50 pM, at least 60 pM, at least 7 pM, at least 80 pM, at least 90 pM, at least 100 pM, at least 200 pM, at least 300 pM, at least 400 pM, at least 500 pM, at least 600 pM, at least 700 pM, at least 800 pM, at least 900 pM, at least 1 nanomolar (nM), at least 2 nM, at least 3 nM, at least 4 nM, at least 5 nM, at least 6 nM, at least 7 nM, at least 8 nM, at least 9 nM, at least 10 nM, at least 20 nM, at least 30 nM, at least 40 nM, at least 50 nm, at least 60 nM, at least 70 nM, at least 80 nM, at least 90 nM, at least 100 nM, at least 200 nM, at least 300 nM, at least 400 nM, at least 500 nM, at least 600 nM, at least 700 nM, at least 800 nM, at least 900 nM, at least 1 μM, at least 2 μM, at least 3 μM, at least 4 μM, at least 5 μM, at least 6 μM, at least 7 μM, at least 8 μM, at least 9 μM, at least 10 μM, at least 20 μM, at least 30 μM, at least 40 μM, at least 50 μM, at least 60 μM, at least 70 μM, at least 80 μM, at least 90 μM, or about 100 μM.

A dynamic range of binding of an antibody from a biological sample to a peptide microarray can be described as the ratio between the largest and smallest value of a detected signal of binding. A signal of binding can be, for example, a fluorescent signal detected with a secondary antibody. Traditional assays are limited by pre-determined and narrow dynamic ranges of binding. The methods and arrays of the disclosure can detected a broad dynamic range of antibody binding to the peptides in the array. In some embodiments, a broad dynamic range of antibody binding can be detected on a logarithmic scale. In some embodiments, the methods and arrays of the disclosure allow the detection of a pattern of binding of a plurality of antibodies to an array using up to 2 logs of dynamic range, up to 3 logs of dynamic range, up to 4 logs of dynamic range or up to 5 logs of dynamic range.

Once the peptides bound by antibody are determined, the method further includes translating the signature peptide profile to high affinity peptides for an antibody of interest, wherein the presence of the antibody of interest identifies a subject as having the a disease or condition of interest, for example to create a diagnostic peptide array for one or multiple diseases or conditions. There are multiple approaches to constructing a set of high affinity peptides that bind an antibody of interest. In one embodiment, one or more antibodies of interest are affinity purified using the random peptides they bind, for example using the differentially bound peptides from the peptide array. In some embodiments, one or more antibodies of interest reverse engineer the antibody using a peptide sequence, which may be graphed, for example by nucleic acid manipulation into an antibody scaffold. The antibody can then be used to probe the relevant cellular material to find the original antigen target, for example, by combining a pull down or Western blot with mass spectrometry. Once the target has been found, it can be used to determine the actual peptide sequence that induced the one or more antibodies of interest. In embodiments, a peptide, for example, a mimotope, is derived that has higher affinity for the informative antibody than an informative random peptide from the array. This can be done by creating a library of variants on one or more antibodies of interest and selecting or screening for those with the highest affinity. This sequence does not have to correspond to the natural sequence of the inducing antigen, but is high affinity for the informative antibody. An advantage of this method is that the target antigen and/or epitope does not need to be identified. In embodiments, a peptide, for example, that has high affinity for the informative antibody is determined by informatic induction. For example, by aligning the sequences in the informative peptides potential epitope targets can be identified. These potential epitope targets can be matched against proteome sequences to find the original antigen and the natural high affinity peptide identified. Alternatively, the sequences of the potential epitope targets can be used as described above to define a high affinity peptide. However, in all of the methods described the goal is to assemble a collection of peptides that would bind all the antibodies that formed the original set of high affinity peptides for detecting one or more antibodies that are associated with one or more diseases or conditions of interest. These peptides would be high affinity to the informative antibody.

Once the high affinity peptides for the antibody of interest are identified they can be used to create a diagnostic peptide array. Any number of peptides or disease and conditions may be used or detected with such a peptide array. In certain embodiments, each one of the peptides in the diagnostic array specifically binds an antibody with high affinity.

Any suitable peptide array can be used for an array on which the peptides are immobilized to a substrate, including informative arrays and diagnostic arrays. In some embodiments, the array comprises between 500-1,000,000 peptides; between 500-500,000 peptides; between 500-250,000 peptides; between 500-100,000 peptides; between 500-50,000 peptides; or between 500-10,000 peptides. In some embodiments, the peptides are 8-35, 12-35, 15-25, 10-30, or 9-25 amino acids in length. In in the case of peptide arrays used to identify the set of high affinity peptides for detecting one or more antibodies that are associated with one or more diseases or conditions of interest in some embodiments, the amino acid sequences of the peptides are randomly selected, for example from random sequence space. The use of random sequence peptides enables the diversity of the antibody repertoire to be matched by an unbiased, comprehensive library of ligands to screen. Since random peptide sequences have no constraints and no intentional homology to biological space, the microarrays contain sparse but very broad coverage of sequence space. Normal, mutated, post-translationally modified, and mimetic epitopes corresponding to any disease or organism can be screened on the same microarray. In some embodiments however, the peptide arrays used to identify the set of high affinity peptides for detecting one or more antibodies that are associated with one or more diseases or conditions of interest in some embodiments, the pattern of amino acids present in the array is pre-defined, and is not a random peptide array. With respect to the disclosed diagnostic peptide arrays, the pattern of amino acids present in the array is pre-defined, and the array is not a random peptide array.

As used herein, the term “substrate” refers to any type of solid support to which the peptides are immobilized. Examples of substrates include, but are not limited to, microarrays; beads; columns; optical fibers; wipes; nitrocellulose; nylon; glass; quartz; diazotized membranes (paper or nylon); silicones; polyformaldehyde; cellulose; cellulose acetate; paper; ceramics; metals; metalloids; semiconductive materials; coated beads; magnetic particles; plastics such as polyethylene, polypropylene, and polystyrene; gel-forming materials; silicates; agarose; polyacrylamides; methylmethacrylate polymers; sol gels; porous polymer hydrogels; nanostructured surfaces; nanotubes (such as carbon nanotubes); and nanoparticles (such as gold nanoparticles or quantum dots). When bound to a substrate, the peptides can be directly linked to the support, or attached to the surface via a linker. Thus, the solid substrate and/or the peptides can be derivatized using methods known in the art to facilitate binding of the peptides to the solid support, so long as the derivatization does not eliminate detection of binding between the peptides and an antibody.

Other molecules, such as reference or control molecules, can be optionally immobilized on the substrate as well. Methods for immobilizing various types of molecules on a variety of substrates are well known to those of skill in the art. A wide variety of materials can be used for the solid surface. A variety of different materials can be used to prepare the support to obtain various properties. For example, proteins (e.g., bovine serum albumin) or mixtures of macromolecules (e.g., Denhardt's solution) can be used to minimize non-specific binding, simplify covalent conjugation, and/or enhance signal detection.

The peptide arrays can be contacted with a biological sample under any suitable conditions to promote binding of antibodies in the biological sample to peptides immobilized on the array. Thus, the disclosed methods are not limited by any specific type of binding conditions employed. Such conditions will vary depending on the array being used, the type of substrate, the density of the peptides arrayed on the substrate, desired stringency of the binding interaction, and nature of the competing materials in the binding solution. In some embodiments, the conditions comprise a step to remove unbound antibodies from the addressable array. Determining the need for such a step, and appropriate conditions for such a step, are well within the level of skill in the art.

Similarly, any suitable detection technique can be used in the disclosed methods detecting binding of antibodies in the biological sample to peptides on the array to generate a disease immune profile; In one embodiment, any type of detectable label can be used to label peptides on the array, including but not limited to radioisotope labels, fluorescent labels, luminescent labels, and electrochemical labels (i.e.: ligand labels with different electrode mid-point potential, where detection comprises detecting electric potential of the label). Alternatively, bound antibodies can be detected, for example, using a detectably labeled secondary antibody.

A peptide array can comprise a plurality of different peptides patterns a surface. A peptide array can comprise, for example, a single, a duplicate, a triplicate, a quadruplicate, a quintuplicate, a sextuplicate, a septuplicate, an octuplicate, a nonuplicate, and/or a decuplicate replicate of the different pluralities of peptides and/or molecules. In some embodiments, pluralities of different peptides are spotted in replica on the surface of a peptide array. A peptide array can, for example, comprise a plurality of peptides homogenously distributed on the array. A peptide array can, for example, comprise a plurality of peptides heterogeneously distributed on the array.

An intra-amino acid distance in a peptide array is the distance between each peptide in a peptide microarray. An intra-amino acid distance can contribute to an off-target binding and/or to an avidity of binding of an antibody to an array. An intra-amino acid difference can be about 0.5 nm, about 1 nm, about 1 nm, 1.1 nm, about 1.2 nm, about 1.3 nm, about 1.4 nm, about 1.5 nm, about 1.6 nm, about 1.7 nm, about 1.8 nm, about 1.9 nm, about 2 nm, about 2.1 nm, about 2.2 nm, about 2.3 nm, about 2.4 nm, about 2.5 nm, about 2.6 nm, about 2.7 nm, about 2.8 nm, about 2.9 nm, about 3 nm, about 3.1 nm, about 3.2 nm, about 3.3 nm, about 3.4 nm, about 3.5 nm, about 3.6 nm, about 3.7 nm, about 3.8 nm, about 3.9 nm, about 4 nm, about 4.1 nm, about 4.2 nm, about 4.3 nm, about 4.4 nm, about 4.5 nm, about 4.6 nm, about 4.7 nm, about 4.8 nm, about 4.9 nm, about 5 nm, about 5.1 nm, about 5.2 nm, about 5.3 nm, about 5.4 nm, about 5.5 nm, about 5.6 nm, about 5.7 nm, about 5.8 nm, about 5.9 nm, and/or about 6 nm. In some embodiments, the intra-amino acid distance is less than 6 nanometers (nm).

An intra-amino acid difference can be at least 0.5 nm, at least 1 nm, at least 1 nm, at least 1.1 nm, at least 1.2 nm, at least 1.3 nm, at least 1.4 nm, at least 1.5 nm, at least 1.6 nm, at least 1.7 nm, at least 1.8 nm, at least 1.9 nm, at least 2 nm, at least 2.1 nm, at least 2.2 nm, at least 2.3 nm, at least 2.4 nm, at least 2.5 nm, at least 2.6 nm, at least 2.7 nm, at least 2.8 nm, at least 2.9 nm, at least 3 nm, at least 3.1 nm, at least 3.2 nm, at least 3.3 nm, at least 3.4 nm, at least 3.5 nm, at least 3.6 nm, at least 3.7 nm, at least 3.8 nm, at least 3.9 nm, at least 4 nm, at least 4.1 nm, at least 4.2 nm, at least 4.3 nm, at least 4.4 nm, at least 4.5 nm, at least 4.6 nm, at least 4.7 nm, at least 4.8 nm, at least 4.9 nm, at least 5 nm, at least 5.1 nm, at least 5.2 nm, at least 5.3 nm, at least 5.4 nm, at least 5.5 nm, at least 5.6 nm, at least 5.7 nm, at least 5.8 nm, or at least 5.9 nm.

An intra-amino acid difference can be not more than 0.5 nm, not more than 1 nm, not more than 1 nm, not more than 1.1 nm, not more than 1.2 nm, not more than 1.3 nm, not more than 1.4 nm, not more than 1.5 nm, not more than 1.6 nm, not more than 1.7 nm, not more than 1.8 nm, not more than 1.9 nm, not more than 2 nm, not more than 2.1 nm, not more than 2.2 nm, not more than 2.3 nm, not more than 2.4 nm, not more than 2.5 nm, not more than 2.6 nm, not more than 2.7 nm, not more than 2.8 nm, not more than 2.9 nm, not more than 3 nm, not more than 3.1 nm, not more than 3.2 nm, not more than 3.3 nm, not more than 3.4 nm, not more than 3.5 nm, not more than 3.6 nm, not more than 3.7 nm, not more than 3.8 nm, not more than 3.9 nm, not more than 4 nm, not more than 4.1 nm, not more than 4.2 nm, not more than 4.3 nm, not more than 4.4 nm, not more than 4.5 nm, not more than 4.6 nm, not more than 4.7 nm, not more than 4.8 nm, not more than 4.9 nm, not more than 5 nm, not more than 5.1 nm, not more than 5.2 nm, not more than 5.3 nm, not more than 5.4 nm, not more than 5.5 nm, not more than 5.6 nm, not more than 5.7 nm, not more than 5.8 nm, not more than 5.9 nm, and/or not more than 6 nm. In some embodiments, the intra-amino acid distance is not more than 6 nanometers (nm).

An intra-amino acid difference can range from 0.5 nm to 1 nm, 0.5 nm to 2 nm, 0.5 nm to 3 nm, 0.5 nm to 3 nm, 0.5 nm to 4 nm, 0.5 nm to 5 nm, 0.5 nm to 6 nm, 1 nm to 2 nm, 1 nm to 3 nm, 1 nm to 4 nm, 1 nm to 5 nm, 1 nm to 6 nm, 2 nm to 3 nm, 2 nm to 4 nm, 2 nm to 5 nm, 2 nm to 6 nm, 3 nm to 4 nm, 3 nm to 5 nm, 3 nm to 6 nm, 4 nm to 5 nm, 4 nm to 6 nm, and/or 5 nm to 6 nm.

A peptide array can comprise a plurality of different peptides patterns a surface. A peptide array can comprise, for example, a single, a duplicate, a triplicate, a quadruplicate, a quintuplicate, a sextuplicate, a septuplicate, an octuplicate, a nonuplicate, and/or a decuplicate replicate of the different pluralities of peptides and/or molecules. In some embodiments, pluralities of different peptides are spotted in replica on the surface of a peptide array. A peptide array can, for example, comprise a plurality of peptides homogenously distributed on the array. A peptide array can, for example, comprise a plurality of peptides heterogeneously distributed on the array.

A peptide can be “spotted” in a peptide array. A peptide spot can have various geometric shapes, for example, a peptide spot can be round, square, rectangular, and/or triangular. A peptide spot can have a plurality of diameters. Non-limiting examples of peptide spot diameters are about 3 μm to about 8 μm, about 3 to about 10 mm, about 5 to about 10 mm, about 10 μm to about 20 μm, about 30 μm, about 40 μm, about 50 μm, about 60 μm, about 70 μm, about 80 μm, about 90 μm, about 100 μm, about 110 μm, about 120 μm, about 130 μm, about 140 μm, about 150 μm, about 160 μm, about 170 μm, about 180 μm, about 190 μm, about 200 μm, about 210 μm, about 220 μm, about 230 μm, about 240 μm, and/or about 250 μm

A peptide array can comprise a number of different peptides. In some embodiments, a peptide array comprises about 10 peptides, about 50 peptides, about 100 peptides, about 200 peptides, about 300 peptides, about 400 peptides, about 500 peptides, about 750 peptides, about 1000 peptides, about 1250 peptides, about 1500 peptides, about 1750 peptides, about 2,000 peptides; about 2,250 peptides; about 2,500 peptides; about 2,750 peptides; about 3,000 peptides; about 3,250 peptides; about 3,500 peptides; about 3,750peptides; about 4,000 peptides; about 4,250 peptides; about 4,500 peptides; about 4,750 peptides; about 5,000 peptides; about 5,250 peptides; about 5,500 peptides; about 5,750 peptides; about 6,000 peptides; about 6,250 peptides; about 6,500 peptides; about 7,500 peptides; about 7,725 peptides 8,000 peptides; about 8,250 peptides; about 8,500 peptides; about 8,750 peptides; about 9,000 peptides; about 9,250 peptides; about 10,000 peptides; about 10,250 peptides; about 10,500 peptides; about 10,750 peptides; about 11,000 peptides; about 11,250 peptides; about 11,500 peptides; about 11,750 peptides; about 12,000 peptides; about 12,250 peptides; about 12,500 peptides; about 12,750 peptides; about 13,000 peptides; about 13,250 peptides; about 13,500 peptides; about 13,750 peptides; about 14,000 peptides; about 14,250 peptides; about 14,500 peptides; about 14,750 peptides; about 15,000 peptides; about 15,250 peptides; about 15,500 peptides; about 15,750 peptides; about 16,000 peptides; about 16,250 peptides; about 16,500 peptides; about 16,750 peptides; about 17,000 peptides; about 17,250 peptides; about 17,500 peptides; about 17,750 peptides; about 18,000 peptides; about 18,250 peptides; about 18,500 peptides; about 18,750 peptides; about 19,000 peptides; about 19,250 peptides; about 19,500 peptides; about 19,750 peptides; about 20,000 peptides; about 20,250 peptides; about 20,500 peptides; about 20,750 peptides; about 21,000 peptides; about 21,250 peptides; about 21,500 peptides; about 21,750 peptides; about 22,000 peptides; about 22,250 peptides; about 22,500 peptides; about 22,750 peptides; about 23,000 peptides; about 23,250 peptides; about 23,500 peptides; about 23,750 peptides; about 24,000 peptides; about 24,250 peptides; about 24,500 peptides; about 24,750 peptides; about 25,000 peptides; about 25,250 peptides; about 25,500 peptides; about 25,750 peptides; and/or about 30,000 peptides.

In some embodiments, a peptide array used in the methods and devices herein comprises more than 30,000 peptides. In some embodiments, a peptide array used in a method of health monitoring comprises about 330,000 peptides. In some embodiments the array comprise about 30,000 peptides; about 35,000 peptides; about 40,000 peptides; about 45,000 peptides; about 50,000 peptides; about 55,000 peptides; about 60,000 peptides; about 65,000 peptides; about 70,000 peptides; about 75,000 peptides; about 80,000 peptides; about 85,000 peptides; about 90,000 peptides; about 95,000 peptides; about 100,000 peptides; about 105,000 peptides; about 110,000 peptides; about 115,000 peptides; about 120,000 peptides; about 125,000 peptides; about 130,000 peptides; about 135,000 peptides; about 140,000 peptides; about 145,000 peptides; about 150,000 peptides; about 155,000 peptides; about 160,000 peptides; about 165,000 peptides; about 170,000 peptides; about 175,000 peptides; about 180,000 peptides; about 185,000 peptides; about 190,000 peptides; about 195,000 peptides; about 200,000 peptides; about 210,000 peptides; about 215,000 peptides; about 220,000 peptides; about 225,000 peptides; about 230,000 peptides; about 240,000 peptides; about 245,000 peptides; about 250,000 peptides; about 255,000 peptides; about 260,000 peptides; about 265,000 peptides; about 270,000 peptides; about 275,000 peptides; about 280,000 peptides; about 285,000 peptides; about 290,000 peptides; about 295,000 peptides; about 300,000 peptides; about 305,000 peptides; about 310,000 peptides; about 315,000 peptides; about 320,000 peptides; about 325,000 peptides; about 330,000 peptides; about 335,000 peptides; about 340,000 peptides; about 345,000 peptides; and/or about 350,000 peptides. In some embodiments, a peptide array used in a method of health monitoring comprises more than 330,000 peptides.

A peptide array can comprise a number of different peptides. In some embodiments, a peptide array comprises at least 2,000 peptides; at least 3,000 peptides; at least 4,000 peptides; at least 5,000 peptides; at least 6,000 peptides; at least 7,000 peptides; at least 8,000 peptides; at least 9,000 peptides; at least 10,000 peptides; at least 11,000 peptides; at least 12,000 peptides; at least 13,000 peptides; at least 14,000 peptides; at least 15,000 peptides; at least 16,000 peptides; at least 17,000 peptides; at least 18,000 peptides; at least 19,000 peptides; at least 20,000 peptides; at least 21,000 peptides; at least 22,000 peptides; at least 23,000 peptides; at least 24,000 peptides; at least 25,000 peptides; at least 30,000 peptides; at least 40,000 peptides; at least 50,000 peptides; at least 60,000 peptides; at least 70,000 peptides; at least 80,000 peptides; at least 90,000 peptides; at least 100,000 peptides; at least 110,000 peptides; at least 120,000 peptides; at least 130,000 peptides; at least 140,000 peptides; at least 150,000 peptides; at least 160,000 peptides; at least about 170,000 at least 180,000 peptides; at least 190,000 peptides; at least 200,000 peptides; at least 210,000 peptides; at least 220,000 peptides; at least 230,000 peptides; at least 240,000 peptides; at least 250,000 peptides; at least 260,000 peptides; at least 270,000 peptides; at least 280,000 peptides; at least 290,000 peptides; at least 300,000 peptides; at least 310,000 peptides; at least 320,000 peptides; at least 330,000 peptides; at least 340,000 peptides; at least 350,000 peptides. In some embodiments, a peptide array used in a method of health monitoring comprises at least 330,000 peptides.

A peptide can be physically tethered to a peptide array by a linker molecule. The N- or the C-terminus of the peptide can be attached to a linker molecule. A linker molecule can be, for example, a functional plurality or molecule present on the surface of an array, such as an imide functional group, an amine functional group, a hydroxyl functional group, a carboxyl functional group, an aldehyde functional group, and/or a sulfhydryl functional group. A linker molecule can be, for example, a polymer. In some embodiments the linker is maleimide. In some embodiments the linker is a glycine-serine-cysteine (GSC) or glycine-glycine-cysteine (GGC) linker. In some embodiments, the linker consists of a polypeptide of various lengths or compositions. In some cases the linker is polyethylene glycol of different lengths. In yet other cases, the linker is hydroxymethyl benzoic acid, 4-hydroxy-2-methoxy benzaldehyde, 4-sulfamoyl benzoic acid, or other suitable for attaching a peptide to the solid substrate.

A surface of a peptide array can comprise a plurality of different materials. A surface of a peptide array can be, for example, glass. Non-limiting examples of materials that can comprise a surface of a peptide array include glass, functionalized glass, silicon, germanium, gallium arsenide, gallium phosphide, silicon dioxide, sodium oxide, silicon nitrade, nitrocellulose, nylon, polytetrafluoroethylene, polyvinylidenedifluoride, polystyrene, polycarbonate, methacrylates, or combinations thereof.

A surface of a peptide array can be flat, concave, or convex. A surface of a peptide array can be homogeneous and a surface of an array can be heterogeneous. In some embodiments, the surface of a peptide array is flat.

A surface of a peptide array can be coated with a coating. A coating can, for example, improve the adhesion capacity of an array. A coating can, for example, reduce background adhesion of a biological sample to an array. In some embodiments, a peptide array of comprises a glass slide with an aminosilane-coating.

A peptide array can have a plurality of dimensions. A peptide array can be a peptide microarray.

Binding interactions between components of a sample and an array can be detected in a variety of formats. In some formats, components of the samples are labeled. The label can be a radioisotype or dye among others. The label can be supplied either by administering the label to a patient before obtaining a sample or by linking the label to the sample or selective component(s) thereof.

Binding interactions can also be detected using a secondary detection reagent, such as an antibody. For example, binding of antibodies in a sample to an array can be detected using a secondary antibody specific for the isotype of an antibody (e.g., IgG (including any of the subtypes, such as IgG1, IgG2, IgG3 and IgG4), IgA, IgM). The secondary antibody is usually labeled and can bind to all antibodies in the sample being analyzed of a particular isotype. Different secondary antibodies can be used having different isotype specificities. Although there is often substantial overlap in compounds bound by antibodies of different isotypes in the same sample, there are also differences in profile.

Binding interactions can also be detected using label-free methods, such as surface plasmon resonance (SPR) and mass spectrometry. SPR can provide a measure of dissociation constants, and dissociation rates. The A-100 Biocore/GE instrument, for example, is suitable for this type of analysis. FLEXchips can be used to analyze up to 400 binding reactions on the same support.

Disclosed are peptide arrays produced by the methods described herein as well as kits including such peptide arrays.

### Methods of Detecting a Disease or Condition

The present disclosure provides peptides arrays for the use of medical diagnostics, for example to detect a disease and/or condition in sample, such as a sample obtained from a subject. In some embodiments, the peptide array may be used in determining response to administration of drugs or vaccines. In embodiments, a method of detecting an antibody associated with a disease of condition, incudes contacting a biological sample with a diagnostic peptide array, and detecting the binding of one or more antibodies to a peptide that is associated with a disease or condition of interest, thereby detecting the antibody associated with a disease of condition. A condition that can be diagnosed or prognosed with a peptide array includes, for example, cancer, autoimmune disorder, an infectious disease, an epidemic, transplant rejection, a metabolic disease, a cardiovascular disease, a dermatological disease, a hematological disease, a neurodegenerative disease, an inflammatory disease, and infarctions (e.g. myocardial infarction, stroke).

In some embodiments, the binding of a molecule to an array as disclosed herein creates a pattern of binding that can be associated with a condition. The affinity of binding of a molecule to a peptide in the array can be mathematically associated with a condition. The off-target binding pattern of an antibody to a plurality of different peptides of embodiments provided herein can be mathematically associated with a condition. The avidity of binding of a molecule to a plurality of different peptides of embodiments provided herein can be mathematically associated with a condition. The off-target binding and avidity can comprise the interaction of a molecule in a biological sample with multiple, non-identical peptides in a peptide array. An avidity of binding of a molecule with multiple, non-identical peptides in a peptide array can determine an association constant of the molecule to the peptide array. In some embodiments, the concentration of an antibody in a sample contributes to an avidity of binding to a peptide array, for example, by trapping a critical number or antibodies in the array and allowing for rapid rebinding of an antibody to an array.

A diagnostic peptide array of the present disclosure can be used to diagnose or prognose cancers including, for example, prostate cancer, lung cancer, colon cancer, bladder cancer, brain cancer, breast cancer, esophageal cancer, Hodgkin lymphoma, kidney cancer, larynx cancer, leukemia, liver cancer, melanoma of the skin, myeloma, non-Hodgkin lymphoma, oral cavity cancer, ovarian cancer, pancreatic cancer, rectal cancer, stomach cancer, testicular cancer, thyroid cancer, urinary bladder cancer, and cervical cancer.

A diagnostic peptide array of the present disclosure can be used to diagnose or prognose cancers including epidemics caused by, for example, viruses, bacteria, or parasites, or non-infectious agents.

A diagnostic peptide array of the present disclosure can be used to diagnose or prognose metabolic disease including, for example, abetalipoproteinemia, adrenoleukodystrophy (ALD), crigler-najjar syndrome, cystinuria, hartnup disease, histidinemia, Menkes disease, phenylketonuria (PKU), sitosterolemia, Smith-Lemli-Opiz syndrome, tyrosinemia type I, urea cycle disorders, Wilson's disease, Zellweger syndrome, maple syrup urine disease (MSUD; branched-chain ketoaciduria), glycogen storage disease, glutaric acidemia type 1, alcaptonuria, medium chain acyl dehydrogenase deficiency (glutaric acidemia type 2), acute intermittent porphyria, Lesch-Nhyhan syndrome, congenital adrenal hyperplasia, Kearns-Sayre syndrome, Gaucher's disease, diabetes (type 1), hereditary hemochromatosis, and Niemann-Pick disease.

A diagnostic peptide array of the present disclosure can be used to diagnose or prognose cardiovascular disease including, for example, angina, arrhythmia, atherosclerosis, cardiomyopathy, cerebrovascular accident (stroke), cerebrovascular disease, congenital heart disease, Jye Berghofer Syndrome, congestive heart failure, myocarditis, valve disease, coronary artery disease, dilated cardiomyopathy, diastolic dysfunction, endocarditis, high blood pressure (hypertension), hypertrophic cardiomyopathy, mitral valve prolapse, myocardial infarction, venous thromboembolism.

A diagnostic peptide array of the present disclosure can be used to diagnose or prognose dermatological disorders including, for example, acne, actinic keratosis, angioma, Athlete's foot, aquagenic pruritus, argyria, atopic dermatitis, baldness, basal cell carcinoma, bed sore, Bechet's disease, blepharitis, boil, Bowen's disease, bullous pemphigoid, canker sore, carbuncles, cellulitis, chloracne, chronic dermatitis of the hands and feet, cold sores, contact dermatitis (includes poison ivy, oak, sumac), creeping eruption, dandruff, dermatitis, dermatitis herpetiformis, dermatofibroma, diaper rash, dyshidrosis, eczema, epidermolysis bullosa, erysipelas, erythroderma, friction blister, genital wart, gestational pemphigoid, Grover's disease, hemangioma, Hidradenitis suppurativa, hives, hyperhidrosis, ichthyosis, impetigo, jock itch, Kaposi's sarcoma, keloid, keratoacanthoma, keratosis pilaris, Lewandowsky-Lutz dysplasia, lice infection, Lichen planus, Lichen simplex chronicus, lipoma, lymphadenitis, malignant melanoma, melasma, miliaria, molluscum contagiosum, nummular dermatitis, Paget's disease of the nipple, pediculosis, pemphigus, perioral dermatitis, photoallergy, photosensitivity, Pityriasis rosea, Pityriasis rubra pilaris, porphyria, psoriasis, Raynaud's disease, ringworm, rosacea, scabies, scleroderma, scrofula, sebaceous cyst, seborrheic keratosis, seborrhoeic dermatitis, shingles, skin cancer, skin tags, spider veins, squamous cell carcinoma, stasis dermatitis, sunburn, tick bite, tinea barbae, tinea capitis, tinea corporis, tinea cruris, tinea pedis, tinea unguium, tinea versicolor, tinea, tungiasis, urticaria (Hives), Vagabond's disease, vitiligo, warts, wheal (“weal” and “welt”).

A diagnostic peptide array of the present disclosure can be used to diagnose or prognose hematological disorders including, for example Anaphylactoid Purpura (Henock-Schönlein Disease), allergic purpura (Henock-Schönlein Disease), low red blood cells (anemia), hemolytic anemia, hypoproliferative anemia, macrocytic anemia, microcytic anemia, normocytic anemia, pernicious anemia (Vitamin B12 deficiency), basophilia, blood vessel abnormalities, dysfibrinogenemia, eosinophilia, erythrocytosis/polycythemia, essential thrombocythemia, excess platelets (thrombocytosis), excess red blood cells (erythrocytosis/polycythemia), excess white blood cells (leukocytosis), Factor V Leiden Mutation, fibrin clot formation abnormalities, folic acid deficiency, hemophilia, hereditary von Willebrand's Disease, inherited hypercoagulation disorders, inherited platelet abnormalities, iron deficiency, low platelets (thrombocytopenia), low white blood cells (neutropenia), lymphocytosis, myelofibrosis with myeloid metaplasia, monocytosis, myeloproliferative disorders, neutrophilia, platelet abnormalities, polycythemia vera, premalignant blood disorders, scurvy, Systemic Lupus Erythematosus (SLE), thrombocytopenia, and sickle cell disease.

A diagnostic peptide array of the present disclosure can be used to diagnose or prognose neurodegenerative diseases including, for example, alcoholism, Alexander's disease, Alper's disease, Alzheimer's disease, Amyotrophic lateral sclerosis, ataxia telangiectasia, Batten disease (also known as Spielmeyer-Vogt-Sjogren-Batten disease), bovine spongiform encephalopathy (BSE), Canavan disease, Cockayne syndrome, Corticobasal degeneration, Creutzfeldt-Jakob disease, Huntington's disease, HIV-associated dementia, Kennedy's disease, Krabbe's disease, Lewy body dementia, Machado-Joseph disease (Spinocerebellar ataxia type 3), multiple sclerosis, Multiple System Atrophy, narcolepsy, neuroborreliosis, Parkinson's disease, Pelizaeus-Merzbacher Disease, Pick's disease, primary lateral sclerosis, prion diseases, Refsum's disease, Sandhoff's disease, Schilder's disease, subacute combined degeneration of spinal cord secondary to pernicious anaemia, schizophrenia, spinocerebellar ataxia (multiple types with varying characteristics), spinal muscular atrophy, Steele-Richardson-Olszewski disease, and Tabes dorsalis.

A diagnostic peptide array of the present disclosure can be used to diagnose or prognose inflammatory diseases including, for example, asthma, autoimmune diseases, chronic inflammation, chronic prostatitis, glomerulonephritis, hypersensitivities, inflammatory bowel diseases, pelvic inflammatory disease, reperfusion injury, rheumatoid arthritis, transplant rejection, and vasculitis.

In some embodiments, a method of the disclosure can be used as a method of diagnosing, monitoring, and treating a condition. A method of treating a condition can require the prescription of a therapeutic agent targeted to treat the subject's condition or disease. In some embodiments, a therapeutic agent can be prescribed in a range of from about 1 mg to about 2000 mg; from about 5 mg to about 1000 mg, from about 10 mg to about 500 mg, from about 50 mg to about 250 mg, from about 100 mg to about 200 mg, from about 1 mg to about 50 mg, from about 50 mg to about 100 mg, from about 100 mg to about 150 mg, from about 150 mg to about 200 mg, from about 200 mg to about 250 mg, from about 250 mg to about 300 mg, from about 300 mg to about 350 mg, from about 350 mg to about 400 mg, from about 400 mg to about 450 mg, from about 450 mg to about 500 mg, from about 500 mg to about 550 mg, from about 550 mg to about 600 mg, from about 600 mg to about 650 mg, from about 650 mg to about 700 mg, from about 700 mg to about 750 mg, from about 750 mg to about 800 mg, from about 800 mg to about 850 mg, from about 850 mg to about 900 mg, from about 900 mg to about 950 mg, or from about 950 mg to about 1000 mg.

### Biological Samples

he methods and arrays disclosed herein allow for, for example, methods of identifying detect a disease and/or condition with small quantities of biological samples from a subject. In some embodiments, the biological samples can be used in a disclosed method without further processing and in small quantities. In some embodiments, the biological samples comprise, blood, serum, saliva, sweat, cells, tissues, or any bodily fluid. In some embodiments, about 0.5 nl, about 1 nl, about 2 nl, about 3 nl, about 4 nl, about 5 nl, about 6 nl, about 7 nl, about 8 nl, about 9 nl, about 10 nl, about 11 nl, about 12 nl, about 13 nl, about 14 nl, about 15 nl, about 16 nl, about 17 nl, about 18 nl, about 19 nl, about 20 nl, about 21 nl, about 22 nl, about 23 nl, about 24 nl, about 25 nl, about 26 nl, about 27 nl, about 28 nl, about 29 nl, about 30 nl, about 31 nl, about 32 nl, about 33 nl, about 34 nl, about 35 nl, about 36 nl, about 37 nl, about 38 nl, about 39 nl, about 40 nl, about 41 nl, about 42 nl, about 43 nl, about 44 nl, about 45 nl, about 46 nl, about 47 nl, about 48 nl, about 49 nl, or about 50 nl, about 51 nl, about 52 nl, about 53 nl, about 54 nl, about 55 nl, about 56 nl, about 57 nl, about 58 nl, about 59 nl, about 60 nl, about 61 nl, about 62 nl, about 63 nl, about 64 nl, about 65 nl, about 66 nl, about 67 nl, about 68 nl, about 69 nl, about 70 nl, about 71 nl, about 72 nl, about 73 nl, about 74 nl, about 75 nl, about 76 nl, about 77 nl, about 78 nl, about 79 nl, about 80 nl, about 81 nl, about 82 nl, about 83 nl, about 84 nl, about 85 nl, about 86 nl, about 87 nl, about 88 nl, about 89 nl, about 90 nl, about 91 nl, about 92 nl, about 93 nl, about 94 nl, about 95 nl, about 96 nl, about 97 nl, about 98 nl, about 99 nl, about 0.1, about 0.2 μl, about 0.3 μl, about 0.4 μl, about 0.5 μl, about 0.6 μl. about 0.7 μl, about 0.8 μl, about 0.9 μl, about 1 μl, about 2 μl, about 3 μl, about 4 μl, about 5 μl, about 6 μl, about 7 μl, about 8 μl, about 9 μl, about 10 μl, about 11 μl, about 12 μl, about 13 μl, about 14 μl, about 15 μl, about 16 μl, about 17 μl, about 18 μl, about 19 μl, about 20 μl, about 21 μl, about 22 μl, about 23 μl, about 24 μl, about 25 μl, about 26 μl, about 27 μl, about 28 μl, about 29 μl, about 30 μl, about 31 μl, about 32 μl, about 33 μl, about 34 μl, about 35 μl, about 36 μl, about 37 μl, about 38 μl, about 39 μl, about 40 μl, about 41 μl, about 42 μl, about 43 μl, about 44 μl, about 45 μl, about 46 μl, about 47 μ, about 48 μl, about 49 μl, or about 50 μl of biological samples are required for analysis by an array.

A biological sample from a subject can be for example, collected from a subject and directly contacted with an array as disclosed herein. In some embodiments, the biological sample does not require a preparation or processing step prior to being contacted with an array as disclosed herein. In some embodiments, a dry blood sample from a subject is reconstituted in a dilution step prior to being contacted with an array as disclosed herein. A dilution can provide an optimum concentration of an antibody from a biological sample of a subject for testing according to the methods disclosed herein.

In some embodiments, the disclosed methods require no more than about 0.5 nl to about 50 nl, no more than about 1 nl to about 100 nl, no more than about 1 nl to about 150 nl, no more than about 1 nl to about 200 nl, no more than about 1 nl to about 250 nl, no more than about 1 nl to about 300 nl, no more than about 1 nl to about 350 nl, no more than about 1 nl to about 400 nl, no more than about 1 to about 450 nl, no more than about 5 nl to about 500 nl, no more than about 5 nl to about 550 nl, no more than about 5 nl to about 600 nl, no more than about 5 nl to about 650 nl, no more than about 5 nl to about 700 nl, no more than about 5 nl to about 750 nl, no more than about 5 nl to about 800 nl, no more than about 5 nl to about 850 nl, no more than about 5 nl to about 900 nl, no more than about 5 nl to about 950 nl, no more than about 5 nl to about 1 μl, no more than about 0.5 μl to about 1 μl, no more than about 0.5 μl to about 5 μl, no more than about 1 μl to about 10 μl, no more than about 1 μl to about 20 μl, no more than about 1 μl to about 30 μl, no more than about 1 μl to about 40 μl, or no more than about 1 μl to about 50 μl.

In some embodiments, the methods described herein require at least 0.5 nl to about 50 nl, at least about 1 nl to about 100 nl, at least about 1 nl to about 150 nl, at least about 1 nl to about 200 nl, at least about 1 nl to about 250 nl, at least about 1 nl to about 300 nl, at least about 1 nl to about 350 nl, at least about 1 nl to about 400 nl, at least about 1 to about 450 nl, at least about 5 nl to about 500 nl, at least about 5 nl to about 550 nl, at least about 5 nl to about 600 nl, at least about 5 nl to about 650 nl, at least about 5 nl to about 700 nl, at least about 5 nl to about 750 nl, at least about 5 nl to about 800 nl, at least about 5 nl to about 850 nl, at least about 5 nl to about 900 nl, at least about 5 nl to about 950 nl, at least about 5 nl to about 1 μl, at least about 0.5 μl to about 1 μl, at least about 0.5 μl to about 5 μl, at least about 1 μl to about 10 μl, at least about 1 IA to about 20 μl, at least about 1 μl to about 30 μl, at least about 1 μl to about 40 μl, at least about 1 μl to about 50 μl, or at least 50 μl

A subject can provide a plurality of biological sample, including a solid biological sample, from for example, a biopsy or a tissue. In some embodiments, about 1 mg, about 5 mgs, about 10 mgs, about 15 mgs, about 20 mgs, about 25 mgs, about 30 mgs, about 35 mgs, about 40 mgs, about 45 mgs, about 50 mgs, about 55 mgs, about 60 mgs, about 65 mgs, about 7 mgs, about 75 mgs, about 80 mgs, about 85 mgs, about 90 mgs, about 95 mgs, or about 100 mgs of biological sample are used.

In some embodiments, no more than about 1 mg to about 5 mgs, no more than about 1 mg to about 10 mgs, no more than about 1 mg to about 20 mgs, no more than about 1 mg to about 30 mgs, no more than about 1 mg to about 40 mgs, no more than about 1 mg to about 50 mgs, no more than about 50 mgs to about 60 mgs, no more than about 50 mgs to about 70 mgs, no more than about 50 mgs to about 80 mgs, no more than about 50 mgs to about 90 mgs, no more than about 50 mgs to about 100 mgs of biological sample are used by the methods and arrays disclosed herein.

In some embodiments, at least about 1 mg to about 5 mgs, at least about 1 mg to about 10 mgs, at least about 1 mg to about 20 mgs, at least about 1 mg to about 30 mgs, at least about 1 mg to about 40 mgs, at least about 1 mg to about 50 mgs, at least about 50 mgs to about 60 mgs, at least about 50 mgs to about 70 mgs, at least about 50 mgs to about 80 mgs, at least about 50 mgs to about 90 mgs, at least about 50 mgs to about 100 mgs of biological sample are used by the methods and arrays disclosed herein.

In some embodiments, biological samples from a subject are too concentrated and require a dilution prior to being contacted with an array as described herein. A plurality of dilutions can be applied to a biological sample prior to contacting the sample with an array as described herein. A dilution can be a serial dilution, which can result in a geometric progression of the concentration in a logarithmic fashion. For example, a ten-fold serial dilution can be 1 M, 0.01 M, 0.001 M, and a geometric progression thereof. A dilution can be, for example, a one-fold dilution, a two-fold dilution, a three-fold dilution, a four-fold dilution, a five-fold dilution, a six-fold dilution, a seven-fold dilution, an eight-fold dilution, a nine-fold dilution, a ten-fold dilution, a sixteen-fold dilution, a twenty-five-fold dilution, a thirty-two-fold dilution, a sixty-four-fold dilution, and/or a one-hundred-and-twenty-five-fold dilution.

A biological sample can be derived from a plurality of sources within a subject's body and a biological sample can be collected from a subject in a plurality of different circumstances. A biological sample can be collected, for example, during a routine medical consultation, such as a blood draw during an annual physical examination. A biological sample can be collected during the course of a non-routine consultation, for example, a biological sample can be collected during the course of a biopsy. A subject can also collect a biological sample from oneself, and a subject can provide a biological sample to be analyzed by the methods and systems described herein in a direct-to-consumer fashion. In some embodiments, a biological sample can be mailed to a provider of the methods and arrays as described herein. In some embodiments, a dry biological sample, such as a dry blood sample from a subject on a filter paper, is mailed to a provider of the methods and arrays as described herein.

### Kits

Provided by this disclosure are kits that can be used to diagnose a subject with a particular condition or disease and/or monitor the efficacy of a treatment or reoccurrence. Exemplary kits include at least one disclosed array. The disclosed kits can include instructional materials disclosing means of use of the array in the kit. The instructional materials can be written, in an electronic form (such as a computer diskette or compact disk) or can be visual (such as video files).

In some embodiments, the kit may comprise instructions for use in accordance with any of the methods described herein. The included instructions may comprise a description of administration of the formulation according to any of the methods described herein. The kit may further comprise a description of selecting an individual suitable for treatment based on identifying whether that individual has or is at risk of acquiring a particular condition, including cancer.

The instructions relating to the use generally include information as to dosage, dosing schedule, and route of administration for the intended treatment. The containers may be unit doses, bulk packages (e.g., multi-dose packages) or sub-unit doses. Instructions supplied in some embodiments of the kits described herein are typically written instructions on a label or package insert (e.g., a paper sheet included in the kit), but machine-readable instructions (e.g., instructions carried on a magnetic or optical storage disk) are also acceptable. The label or package insert indicates that the composition is used for a particular condition or disease, including treating cancer. Instructions may be provided for practicing any of the methods described herein.

Embodiments of the kits described herein are in suitable packaging. Suitable packaging includes, but is not limited to, vials, bottles, jars, flexible packaging (e.g., sealed Mylar or plastic bags), and the like. Kits may optionally provide additional components such as interpretive information. Normally, the kit comprises a container and a label or package insert(s) on or associated with the container. Some embodiments provided herein relate to articles of manufacture comprising contents of the kits described above.

Throughout this application, various publications are referenced. The disclosures of these publications in their entireties are hereby incorporated by reference into this application in order to more fully describe the state of the art to which this disclosure pertains. The references disclosed are also individually and specifically incorporated by reference herein for the material contained in them that is discussed in the sentence in which the reference is relied upon.

The present disclosure is not to be limited in scope by the specific embodiments described herein. Indeed, various modifications of the disclosure in addition to those described herein will become apparent to those skilled in the art from the foregoing description and the accompanying figures. Such modifications are intended to fall within the scope of the appended claims.

What is claimed is:

1. A method of detecting an antibody associated with a disease or condition of interest, comprising:
contacting an immunosignature peptide array with a biological sample from an individual, wherein the immunosignature peptide array comprises peptides that bind to an antibody of interest;
detecting binding of the antibody of interest in the biological sample to the peptides of the immunosignature peptide array, thereby identifying a set of informative peptides that bind to the antibody of interest;
translating the set of informative peptides to one or more high affinity peptides that bind the antibody of interest;
contacting the biological sample with a diagnostic peptide array comprising the set of high affinity peptides; and
detecting binding of antibodies in the biological sample to the set of high affinity peptides of the diagnostic peptide array.

2. The method of claim 1, wherein translating comprises obtaining the antibody of interest by:
reverse-engineering the antibody of interest using the peptide sequence of an informative peptide, or
affinity purifying the antibody of interest using an informative peptide.

3. The method of claim 1, wherein translating comprises probing the biological sample with the antibody of interest to find an original antigen target, and obtaining the peptide sequence of the original antigen target.

4. The method of claim 1, wherein translating comprises creating a library of variants on an informative peptide, and screening binding of the antibody of interest to the variants.

5. The method of claim 1, wherein translating comprises aligning the peptide sequence of an informative peptide against proteome sequences.

6. The method of claim 1, wherein the condition of interest is a cancer, a metabolic disease, a cardiovascular disease, a dermatological disorder, a hematological disorder, a neurodegenerative disease, or an inflammatory disease.

7. The method of claim 1, wherein the diagnostic peptide array further comprises random sequence peptides or peptides with low binding to an informative antibody.

8. A method of creating a diagnostic peptide array for a disease or condition of interest, comprising:
contacting an immunosignature peptide array with a biological sample from an individual, wherein the immunosignature peptide array comprises peptides that bind to an antibody of interest;
detecting binding of the antibody of interest in the biological sample to the peptides of the immunosignature peptide array, thereby identifying a set of informative peptides that bind to the antibody of interest;
translating the set of informative peptides to one or more high affinity peptides that bind the antibody of interest; and
constructing a diagnostic peptide array comprising the set of high affinity peptides.

9. The method of claim 8, wherein translating comprises obtaining the antibody of interest by:
reverse-engineering the antibody of interest using the peptide sequence of an informative peptide, or
affinity purifying the antibody of interest using an informative peptide.

10. The method of claim 8, wherein translating comprises probing the biological sample with the antibody of interest to find an original antigen target, and obtaining the peptide sequence of the original antigen target.

11. The method of claim 8, wherein translating comprises creating a library of variants on an informative peptide, and screening binding of the antibody of interest to the variants.

12. The method of claim 8, wherein translating comprises aligning the peptide sequence of an informative peptide against proteome sequences.

13. The method of claim 8, wherein the condition of interest is a cancer, a metabolic disease, a cardiovascular disease, a dermatological disorder, a hematological disorder, a neurodegenerative disease, or an inflammatory disease.

14. The method of claim 8, wherein the diagnostic peptide array further comprises random sequence peptides or peptides with low binding to an informative antibody.
